(19) United States
(12) Patent Application Publication
(10) Pub. No.: US 2021/0267463 A1
(43) Pub. Date: Sep. 2, 2021

(54) SYSTEM AND METHOD FOR AUTOMATED THERMOGRAPHIC EXAMINATION

(71) Applicant: Higia, Inc., Wilmington New Castle, DE (US)

(72) Inventors: Julián Ríos Cantú, Monterrey (MX); José Antonio Torres González, San Luis Potosi (MX); Raymundo González Leal, Cadereyta Jimenez (MX); Fernando López Martínez, Tuxtla Gutierrez (MX)

(21) Appl. No.: 17/127,881

(22) Filed: Dec. 18, 2020

Related U.S. Application Data
(60) Provisional application No. 62/982,710, filed on Feb. 27, 2020.

(51) Int. Cl. A61B 5/01; G16H 50/20; A61B 5/00; G16H 30/40; G16H 50/30

(52) U.S. Cl. CPC A61B 5/015; G16H 50/20; A61B 5/4312; G16H 30/40; G16H 50/30; A61B 5/0091

(57) ABSTRACT

A system includes: an exterior enclosure defining an interior examination volume; a thermographic camera defining a field of view encompassing a target examination region within the interior examination volume; and a set of lighting elements configured to illuminate reference locations within the interior examination volume. The system also includes computational subsystem is configured to, for each target pose in a series of target poses: illuminate a reference location via the set of lighting elements, the reference location corresponding to the target pose; prompt the patient to locate within the target examination region and to orient relative to the reference location illuminated via the set of lighting elements; and capture a thermographic image of the patient in the target pose. The computational subsystem is further configured to generate a diagnostic assessment for the patient based on the thermographic image of the patient in each target pose.

### CROSS-REFERENCE TO RELATED APPLICATIONS

This application claims the benefit of U.S. Provisional Application No. 62/982,710, filed on 27 Feb. 2020, which is incorporated in its entirety by this reference.

### TECHNICAL FIELD

This invention relates generally to the field of thermographic screening, monitoring, and diagnostics and more specifically to a new and useful system and method for thermographic examination in the field of thermographic screening, monitoring, and diagnostics.

### BRIEF DESCRIPTION OF THE FIGURES

FIG. 1 is a flowchart representation of a method;

FIG. 2 is a schematic representation of a system;

FIG. 3 is a schematic representation of one variation of the system;

FIG. 4 is a schematic representation of one variation of the system;

FIG. 5 is a schematic representation of one variation of the system;

FIG. 6 is a schematic representation of one variation of the system;

FIG. 7 is a schematic representation of one variation of the system; and

FIG. 8 is a schematic representation of one variation of the system.

### DESCRIPTION OF THE EMBODIMENTS

The following description of embodiments of the invention is not intended to limit the invention to these embodiments but rather to enable a person skilled in the art to make and use this invention. Variations, configurations, implementations, example implementations, and examples described herein are optional and are not exclusive to the variations, configurations, implementations, example implementations, and examples they describe. The invention described herein can include any and all permutations of these variations, configurations, implementations, example implementations, and examples.

### 1. Method

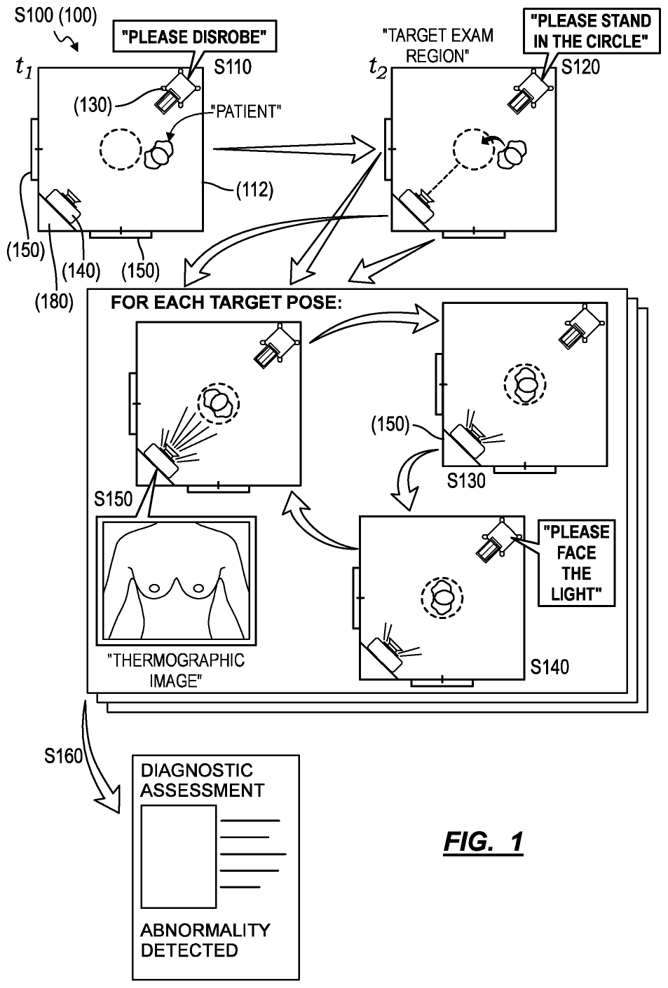

As shown in FIG. 1, a method S100 for administering a thermographic examination is executed at an examination cell 102 including an exterior enclosure 110 defining an interior examination volume, a thermographic camera 140 arranged within the interior examination volume and defining a field of view encompassing a target examination region within the interior examination volume, and a set of lighting elements 150 configured to illuminate a set of reference locations within the interior examination volume. The method S100 includes: at first time, prompting a patient in the interior examination volume to disrobe in Block S110; and at a second time succeeding the first time by an acclimatization duration, prompting the patient to locate to the target examination region in Block S120. The method S100 also includes, for each target pose in a series of target poses: illuminating a reference location in the set of reference locations via the set of lighting elements 150, the reference location corresponding to the target pose in Block S130; prompting the patient to orient relative to the reference location illuminated via the set of lighting elements 150 in Block S140; and capturing a thermographic image of the patient via the thermographic camera 140 in Block S150. The method S100 further includes generating a diagnostic assessment for the patient based on a set of thermographic images comprising the thermographic image of the patient for each target pose in the series of target poses in Block S160.

### 2. System

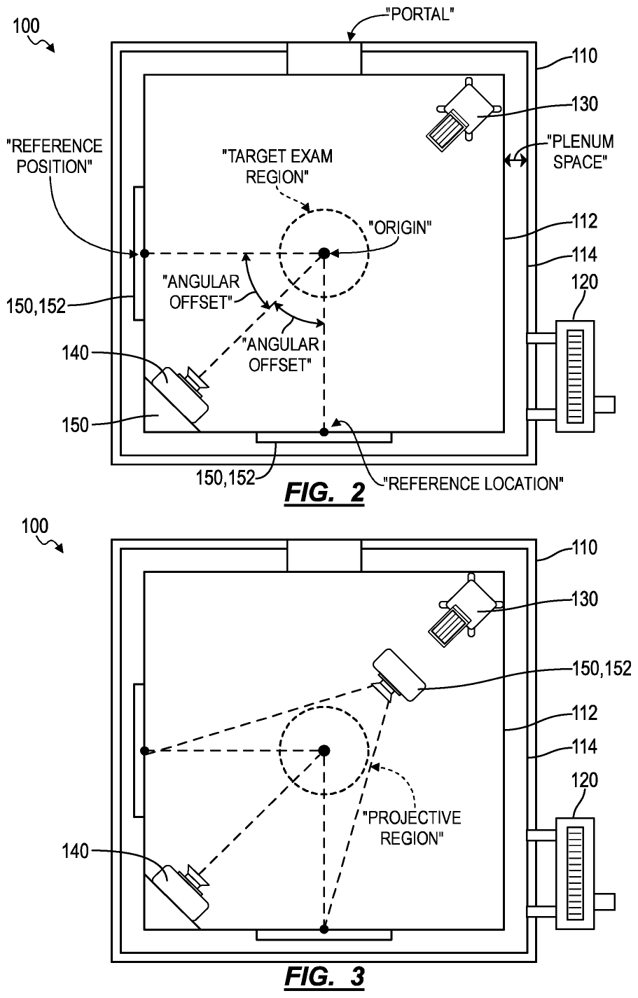

As shown in FIG. 2, a system 100 for thermographic examination includes an examination cell 102 and a computational subsystem 104. The examination cell 102 includes: an exterior enclosure 110 defining an interior examination volume; a thermographic camera 140: arranged within the interior examination volume; and defining a field of view encompassing a target examination region within the interior examination volume; and a set of lighting elements 150 configured to illuminate a set of reference locations within the interior examination volume. The computational subsystem 104 is configured to, following entry of a patient into the interior examination volume, for each target pose in a series of target poses: illuminate a reference location in the set of reference locations via the set of lighting elements 150, the reference location corresponding to the target pose; prompt the patient to locate within the target examination region and to orient relative to the reference location illuminated via the set of lighting elements 150; and capture a thermographic image of the patient in the target pose via the thermographic camera 140. The computational subsystem 104 is further configured to generate a diagnostic assessment for the patient based on a set of thermographic images comprising the thermographic image of the patient in the target pose for each target pose in the series of target poses.

### 3. Applications

Generally, as shown in FIG. 1, the method S100 is executed by a system including an examination cell 102 and a computational subsystem 104 (hereinafter “the system”). The system 100 includes a set of electromechanical components as well as computational devices configured to: fully enclose a patient in a temperature-controlled environment; prompt the patient to position themselves in a series of target poses relative to a thermographic camera 140 via a series of user-experience cues (hereinafter “UX cues”) and/or camera movements; capture a series of thermographic images of the patient in each of the series of target poses; and analyze the series of thermographic images to generate a diagnostic assessment (e.g., a breast cancer risk assessment or a thyroid cancer risk assessment) in near-real-time, all without substantial involvement of a physician or other medical personnel within the examination cell 102. More specifically, as shown in FIG. 2, the examination cell 102 can include: an exterior enclosure 110; a permeable non-reflective (in the infrared spectrum) interior lining 112; a heating, ventilation, and air conditioning subsystem 120 (hereinafter “HVAC subsystem 120”); one or more thermographic cameras 140 arranged within the examination cell 102 configured to record thermographic images of the patient in a series of positions and/or orientations (target poses) relative to the thermographic camera 140; a set of user-experience elements (hereinafter “UX elements”), such as a set of lighting elements 150, configured to guide the patient to assume this series of target poses relative to the thermographic camera 140; and a computational subsystem 104 configured to calculate a diagnostic assessment based on the thermographic images in near-real-time.

Thus, the examination cell 102 can quickly guide a patient through a thermography exam (e.g., a breast thermography exam, a thyroid thermography exam) in accordance with medical best practices without the intervention of medical personnel, while capturing high-quality consistent thermographic images irrespective of patient height, weight, and body type. Additionally, the system 100 can calculate and communicate a diagnostic assessment to the patient in near-real-time (e.g., prior or shortly after the patient leaves the examination cell 102), thereby improving overall user-experience and improving response time for patients with higher indicated risk. Furthermore, the system 100 can be deployed in public spaces outside of medical facilities, which, in combination with the painless and efficient examination process executed by the system 100, may increase the accessibility to breast cancer screening and, therefore, the frequency and consistency with which patients solicit breast examination services.

The system 100 is frequently described herein in the context of breast cancer risk assessment or breast anomaly detection. However, variations of the examination cell 102 can be adapted to detect and/or diagnose other conditions for which thermography is commonly used such as thyroid gland abnormality, vascular disorders, and/or other inflammation producing disorders.

### 3.1 Examples

In one example, a patient arrives at an examination cell 102 located in a mall or other public space. The patient may interface with an operator of the system 100 located outside of the examination cell 102 to make an appointment for a breast examination procedure. Alternatively, the system 100 can be fully automated and the patient can: interface with a touchscreen located outside of the examination cell 102 to make an appointment for a breast examination procedure; or log in to an existing profile associated with the system 100 via a mobile or web application to schedule or confirm a previously existing appointment. Upon confirming an appointment at the examination cell 102, the patient may then enter the examination cell 102 and interface with a user-interface panel or cooperating mobile or web application to create a patient profile and to input patient details, which may include height and weight information as well as previous medical history or log in to a previously existing account already containing this information. Concurrently, the system 100 can prompt the patient to disrobe and adjust the temperature in the examination cell 102 to within an examination range of temperatures to enable the patient to acclimatize to the temperature within the examination cell 102.

Once the patient has completed her patient profile and acclimatized to the temperature within the examination cell 102, the system 100 can initiate an examination procedure by: prompting the user (e.g., via a voice prompts and/or activation of various UX elements) to orient herself relative to the thermographic camera 140 arranged in a corner of the examination cell 102, such as by illuminating a lighting element proximal to the thermographic camera 140 and instructing the patient to face this lighting element. The system 100 can then record a frontal thermographic image of the patient's torso. The system 100 can then prompt the patient to change her orientation relative to the thermographic camera 140, such as by illuminating a lighting element angularly offset from the thermographic camera 140 and instructing the patient to face this newly-illuminated lighting element, and record a second, lateral thermographic image of the patient's torso.

The system 100 can continue to repeat this process for a series of target poses of the patient in order to obtain, for example, a frontal thermographic image (i.e. depicting the patient facing the thermographic camera 140), a first lateral thermographic image (i.e. depicting the patient facing 45 degrees to the left of the thermographic camera 140), and a second lateral thermographic image (i.e. depicting the patient facing 45 degrees to the right of the thermographic camera 140).

Upon completing this above-described examination procedure, the system 100 can instruct or otherwise prompt the patient to dress herself and/or vacate the examination cell 102 while concurrently analyzing the series of thermographic images via a breast cancer diagnostic model. After the patient has dressed herself (or while the patient is dressing herself), the system 100 can communicate a breast cancer risk assessment or other indication of breast health to the user, such as via a user-provided electronic contact method, via a user-interface panel within the examination cell 102, or via an operator of the system 100 located outside of the examination cell 102. In one example, the system 100 can calculate a categorical breast cancer risk assessment, which can communicate that the system 100: has not detected an increased risk of breast cancer in the patient; recommends a further testing by a physician (i.e. there may be an elevated risk of breast cancer in the patient); or recommends a retest at the examination cell 102 (i.e. the ex was inconclusive due to an anomaly). Thus, the system 100 can communicate a categorical breast cancer risk assessment to the patient, as the patient is exiting or preparing to exit the examination cell 102 or shortly thereafter (e.g., within 30 minutes of the examination).

In one example, while adjusting the internal temperature of the examination cell 102, the system 100 can detect heat concentrations (e.g., via the thermographic camera 140) or other temperature anomalies within the examination cell 102 and attempt to correct these anomalies (e.g., via the HVAC subsystem 120) prior to initiating the examination procedure.

In another example, while guiding the patient through the series of target poses and while recording thermographic images of the patient in these target poses, the system 100 can, in real-time: detect whether the patient is in a target pose relative to the thermographic camera 140; and, in response to detecting that the patient is not in the target pose, prompt the user to adjust her position via the set of UX elements within the examination cell 102. Additionally or alternatively, the system 100 can adjust the position of the thermographic camera 140 (e.g., via a gantry system) in order to capture an image of the patient in the target pose relative to the thermographic camera 140.

In yet another example, upon capturing a thermographic image of the patient, the system 100 can verify that the thermographic image satisfies a set of usability criteria, such as exhibiting low levels of blur, low levels of infrared reflection onto the body of the patient, and/or high levels of contrast with the interior lining 112 of the examination cell 102. In response to detecting that one or more of the thermographic images do not satisfy the set of usability criteria, the system 100 can: prompt the patient to reposition herself in the target pose corresponding to the unsatisfactory thermographic image; and record a second thermographic image.

### 4. Examination Cell

Generally, as shown in FIGS. 2-7, the system 100 includes an examination cell 102 including a set of structural components, a set of electromechanical components, and/or a set of computational devices (e.g., tablets, computers, computer processors, computer servers) in communication with each other over a local and/or wide-area network that cooperate with the computational subsystem 104 in order to autonomously administer a thermographic examination procedure (e.g., a thermographic breast examination procedure) for a patient located within the examination cell 102. More specifically, the examination cell 102 can include: an exterior enclosure 110; an intermediate enclosure 114; an interior lining 112; an HVAC subsystem 120; a set of UX elements, which can further include a user-interface panel 130 (hereinafter “UI panel 130”), a set of lighting elements 150, floor indicators, and/or a voice communication subsystem; and/or an imaging subsystem, which can further include a thermographic camera 140 (or a set of thermographic cameras 140), and/or a visible-light camera 142 (or a set of visible-light cameras 142, LIDAR sensors, and/or structured light sensors). Thus, the system 100 can: control the temperature within the examination cell 102; receive relevant personal details and/or a medical history of a patient via the UI panel 130; guide the patient through a series of target poses via the set of UX elements; record thermographic images of the patient in each of these target poses; and calculate a diagnostic assessment, such as a breast cancer risk assessment, based on these images by coordinating the abovementioned components of the examination cell 102 according to Blocks of the method S100.

In one implementation, the examination cell 102 includes the abovementioned components configured in a mobile arrangement (e.g., integrated with a vehicle such as a truck or van such that the examination cell 102 can be easily relocated between examination procedures. Alternatively, the examination cell 102 can be configured as a temporary structure such that the examination cell 102 may be easily: assembled; disassembled; and transported to a new location.

Each of the components of the examination cell 102 is described in further detail below.

### 4.1 Exterior Enclosure

Generally, the examination cell 102 can include an exterior enclosure 110 that operates as the outer shell of the examination cell 102 and insulates the examination cell 102 from the exterior environment. More specifically, the examination cell 102 can include an exterior enclosure no defining an interior examination volume within which a patient can be thermographically examined according to Blocks of the method S100. Thus, the exterior enclosure no structurally supports the examination cell 102 and encloses the interior examination volume to insulate the interior examination volume from the exterior environment and to provide privacy for a patient located within the interior examination volume.

The examination cell 102 can include an exterior enclosure no constructed from an insulating material such as fiberglass, glass, wood, other similar materials, or a combination of these materials. Additionally, the exterior enclosure 110 can define a rectangular, cylindrical, or any other shape similar to an intended configuration of the interior examination volume and can define a portal such as a hinged door or sliding door to allow a patient to enter the examination cell 102. Thus, the exterior enclosure no structurally supports the interior components of the examination cell 102 and insulates the interior of the examination cell 102 to reduce the likelihood of anomalous temperature variations during the examination procedure executed by the examination cell 102.

In one implementation, the exterior enclosure 110 can include a reflective coating or outer layer that reduces absorption of light incident to the examination cell 102, thereby improving the insulating characteristics of the exterior enclosure 110.

In another implementation, the exterior enclosure 110 defines ports and/or mounting points configured to interface with the HVAC subsystem 120 such as an air intake port and an exhaust port as well as a mounting location for the fan, heating element, and/or the heat exchanger of the HVAC subsystem 120.

### 4.2 Intermediate Enclosure

In one implementation, the examination cell 102 can include an intermediate enclosure 114 arranged between the exterior enclosure 110 and the interior lining 112 of the examination cell 102 and defining a thermally insulating layer (of air or insulation material) between the exterior enclosure 110 and the intermediate enclosure 114 and separate this insulating layer from the plenum space between the intermediate enclosure 114 and the interior lining 112 of the examination cell 102. More specifically, the intermediate enclosure 114 can define a similar shape to the exterior enclosure 110 in order to define a thermally insulating layer of a near-constant thickness around the entire surface of the examination cell 102. Additionally, the intermediate enclosure 114 can be manufactured from a similar material to the exterior enclosure 110. Furthermore, the examination cell 102 can include a portal in the intermediate enclosure 114 that is aligned with the portal in the exterior enclosure 110 to enable a patient to enter the interior examination volume of the examination cell 102.

### 4.3 Interior Lining

Generally, the examination cell 102 includes an interior lining 112 that is the patient-facing interior surface of the examination cell 102 and is configured to: maintain a uniform temperature throughout the interior of the examination cell 102; prevent reflection or emission of infrared light onto the patient; and present an appealing and comforting surface to the user. More specifically, the interior lining 112: is arranged within the interior examination volume of the examination cell 102; exhibits low emissivity to prevent infrared projection and/or reflection onto the body of the patient within the examination cell 102; and exhibits sufficient air permeability to enable air to circulate between the interior examination volume of the examination cell 102 and the plenum space between the interior lining 112 and the intermediate enclosure 114. Thus, the interior lining 112 enables the examination cell 102 to accurately maintain the temperature of the interior examination volume without creating temperature gradients within the interior examination volume and to reduce infrared reflections that could result in anomalous thermographic readings on the torso of the patient upon recording thermographic images of the patient.

The interior lining 112 of the examination cell 102 can be constructed from any non-emissive, air-permeable material or combination of materials such a plastic mesh or fabric (e.g., felt or cotton) with cardboard, particle board, or wooden backing. The interior lining 112 exhibits sufficient rigidity to withstand a pressure differential between the plenum space outside of the interior lining 112 and the interior examination space in order to prevent excess bending or deformation of the walls of the interior lining 112. Additionally, the interior lining 112 can define a portal aligned with the portal in the intermediate enclosure 114 and the exterior enclosure 110 to allow entry of a patient into the interior examination volume.

In one implementation, the interior lining 112 defines an interior examination volume with a square footprint in order to facilitate assembly and/or disassembly of the examination cell 102. Alternatively, the interior lining 112 defines an interior examination volume characterized by a circular footprint to distribute infrared reflections from the surface of the interior lining 112 onto the torso of the patient, thereby reducing anomalous temperature data in the thermographic images of the patient's torso. In another alternative, the interior lining 112 can define a teardrop shape, where the patient is directed to stand away from a focus of a smooth side of the teardrop shape in order to minimize infrared reflection onto the patient.

In another implementation, the interior lining 112 is sufficiently translucent to enable the patient to view the set of lighting elements 150 mounted onto the interior surface of the intermediate enclosure 114 and/or onto the exterior surface of the interior lining 112, thereby permitting diffuse light to penetrate the interior lining 112 coincident the positions of the set of lighting elements 150. Alternatively, the interior lining 112 can define bores to enable components of the examination cell 102 to protrude from the interior enclosure into the interior examination volume through the interior lining 112 (e.g., such as lighting elements 150 a projector 154, a thermographic or visible-light camera 142, and/or a UI-panel). In another alternative, the interior lining 112 can also define mounting points about the interior surface of the interior lining 112 that are configured to couple to various other components of the examination cell 102.

In yet another implementation, the interior lining 112 can include a dropped ceiling in order to improve airflow above the dropped ceiling, thereby improving the dissemination of air from a central location (e.g., the location of the HVAC unit) in the ceiling of the examination cell 102 to the sides of the examination cell 102.

### 4.4 Heating Ventilation and Air Conditioning Subsystem

In one implementation, the examination cell 102 can include an HVAC subsystem 120 configured to control the temperature of the interior examination volume of the examination cell 102 to within a predetermined examination range of temperatures (e.g., between 20 and 25 degrees Celsius). More specifically, the examination cell includes an HVAC subsystem 120 configured to force temperature-controlled air into the plenum space; and receive commands from the computational subsystem to control a temperature of the interior examination volume within an examination range of temperatures via the heating, ventilation, and air conditioning subsystem.

In particular, the HVAC subsystem 120 can traverse the exterior enclosure 110 and the intermediate enclosure 114 (via ports in the exterior enclosure no and the intermediate enclosure 114) in order to force cooled and/or heated air into the plenum space between the intermediate enclosure 114 and the interior lining 112 and to pull heated air out of the plenum space. Thus, the HVAC subsystem 120, in cooperation with the interior lining 112, distributes cooled and/or heated air into the interior examination volume of the examination cell 102 without disrupting the airflow within the interior examination volume and, therefore, does not cause temperature swings (e.g., due to evaporative cooling or other effects) on the surface of the patient's torso during execution of the examination process.

In one implementation, the HVAC subsystem 120 can include a standard HVAC unit centrally installed in the ceiling of the examination cell 102 and configured to push cool air into the plenum space between the intermediate enclosure 114 and the interior lining 112 of the examination cell 102. In this implementation, an internal digital thermometer of the HVAC unit that measures the control temperature can be replaced with a digital thermometer located within the interior examination volume. Alternatively, the examination cell 102 can include a set of digital thermometers distributed throughout the interior examination volume and can control an average of these temperatures to within the predetermined examination temperature range.

In another implementation, the examination cell 102 can include an HVAC subsystem 120 installed under the floor of the examination cell 102 and configured to force air upward into the plenum space between the exterior enclosure no and the interior lining 112. In this implementation, the floor of the examination cell 102 can be manufactured from an insulating material to prevent heat from conducting from the HVAC subsystem 120 into the floor of the examination cell 102. Additionally, in this implementation, the examination cell 102 can include an exhaust port to vent heated air produced by the HVAC subsystem 120 out from under the examination cell 102.

In yet another implementation, the HVAC subsystem 120 can include forced exhaust ports that vent air from the perimeter of the plenum space proximal to the ceiling of the intermediate enclosure 114 in order to facilitate more effective cooling of the interior examination volume.

In yet another implementation, the HVAC subsystem 120 can communicate with the computational subsystem 104 in order to modulate the temperature within the interior examination volume at various stages during the examination procedure and/or in response to the detection of temperature anomalies by the examination cell 102.

### 4.4.1 Integrated Black Body Radiator

In one variation, shown in FIG. 7, the examination cell 102 includes a calibrated black body radiator 160 with which the system 100 can calibrate the thermographic camera 140. More specifically, the examination cell 102 can include a wall-integrated black body radiator 160 arranged opposite the thermographic camera 140; and, in this variation, the computational subsystem 104 is configured to capture a calibration image via the thermographic camera 140 depicting the wall-integrated black body radiator 160; and calibrate the thermographic camera 140 based on the calibration image. Thus, the system 100 can automatically calibrate the thermographic camera via the wall-integrated black body radiator 160 that is hidden from the view of a patient located within the interior examination volume.

In one implementation, the examination cell 102 can include a black body radiator 160 mounted to an interior surface of the exterior enclosure 110 or the intermediate enclosure 114. In this implementation, the interior lining can include a window (constructed from a material that is substantially transparent to infrared or near-infrared radiation) that provides a line of sight between the thermographic camera(s) 140 and the black body radiator 160. Alternatively, the system 100 includes a cover configured to open, thereby exposing the wall-integrated black body radiator 160, in coordination with the computational subsystem 104 in order to expose the wall-integrated black body radiator 160 during capture of a thermographic image.

In another implementation, the examination cell 102 can include a wall-integrated black body radiator 160 arranged such that the wall-integrated black body radiator 160 directly contacts the exterior surface of the interior lining 112. Thus, upon activation of the wall-integrated black body radiator 160, the wall-integrated black body radiator 160 heats a region of the interior lining to a target calibration temperature without the wall-integrated black body radiator 160 being visually exposed to a patient within the interior examination volume.

### 4.5 User-Experience Elements

Generally, the examination cell 102 can include a set of UX elements in order to: guide the patient through the examination process and/or receive personal information from the user. More specifically, the examination cell 102 can include a UI panel 130 (e.g., a touchscreen), a set of lighting elements 150 (e.g., wall-integrated and/or projected lighting elements), floor integrated features, and/or a speaker system 100. The system 100 can coordinate each of these UX elements in order to execute Blocks of the method S100 and obtain repeatable and usable thermographic images of patients in the examination cell 102.

In one variation, the set of UX elements includes a set of lighting elements 150 configured to illuminate a set of reference locations within the interior examination volume. In this variation, each reference location in the set of reference locations defines an angular offset between the thermographic camera 140 and the reference location relative to a target examination region (origin position) within the interior examination volume. Thus, a patient may stand or otherwise locate herself at the target examination region and face a reference location illuminated by a subset of the set of lighting elements 150, thereby assuming a target pose relative to the thermographic camera 140.

### 4.5.1 User Interface Panel

Generally, the examination cell 102 can include a UI panel 130 that enables a patient to input personal data and/or her medical history while also providing a visual interface to display information or specific guidance to the patient. More specifically, the examination cell 102 can include a UI panel 130 arranged within the interior examination volume and including a display; and the computational subsystem 104 is configured to, for each target pose in the series of target poses, prompt the patient to locate within the target examination region and to orient relative to the reference location, illuminated via the set of lighting elements 150, via the display of the user-interface panel.

In particular, the UI panel 130 can include a tablet computer, or a display and I/O device mutually connected to a computer, in communication with the computational subsystem 104 of the system 100. The UI panel 130 can be fully integrated with the interior lining 112 of the examination cell 102 (e.g., via a mount or cutout in the interior lining 112). Alternatively, the UI panel 130 is a standalone unit left within the interior examination volume (e.g., on a stand or pedestal within the interior examination volume). The UI panel 130 can communicate with the computational subsystem 104 of the system 100 via a local wireless network or via a wired connection.

### 4.5.2 Wall-Integrated Lighting Elements

Generally, as shown in FIG. 2, the examination cell 102 can include a set of lighting elements 150, such as a set of wall-integrated lighting elements 152, in order to draw the attention of the patient to reference locations in the examination cell 102 that aid the patient in orienting herself (in a target pose) relative to the thermographic camera 140 in preparation for capture of a thermographic image by the system 100. More specifically, the set of wall-integrated lighting elements 152 can include a set of light-emitting diodes (or another low-power low-heat-generating light source) integrated with the interior lining 112 of the examination cell 102 or installed behind a translucent interior lining 112 such that the wall-integrated lighting elements 152 themselves may not be visible to the patient. Therefore, the system 100 can selectively illuminate subsets of the set of lighting elements 150 corresponding to each target pose in the series of target poses at each phase of the thermographic examination process and instruct the patient to orient herself relative to the subset of illuminated lighting elements 150, thereby guiding the patient into the series of target poses relative to the thermographic camera 140 of the examination cell 102.

Thus, the examination cell 102 can include a set of wall-integrated lighting elements 152 arranged at the set of reference locations and integrated with the interior lining 112 beneath an interior surface of the interior lining 112; and the computational subsystem 104 can be configured to, for each target pose in the series of target poses, illuminate the reference location in the set of reference locations by activating a subset of wall-integrated lighting elements 152 in the set of wall-integrated lighting elements 152, the subset of wall-integrated lighting elements 152 arranged at the reference location.

In one implementation, the examination cell 102 includes three subsets of wall-integrated lighting elements 152, each subset of wall-integrated lighting elements 152 corresponding to a reference location in the set of reference locations. In this implementation, a first subset of wall-integrated lighting elements 152 (i.e. a frontal subset of wall-integrated lighting elements 152), is aligned with the thermographic camera 140. Thus, when the examination cell 102 illuminates this first subset of wall-integrated lighting elements 152 and the patient is instructed to face the first subset of illuminated wall-integrated lighting elements 152, a patient following these instructions will face toward the thermographic camera 140. In one example, the first subset of wall-integrated lighting elements 152 can include a recessed strip of wall-integrated lighting elements 152 arranged at a corner between two walls of the examination cell 102 where the thermographic camera 140 is also arranged. In this implementation, the examination cell 102 includes a second subset of wall-integrated lighting elements 152 and a third subset of wall-integrated lighting elements 152 (i.e. a first subset of lateral lighting elements and a second subset of lateral lighting elements) arranged at predetermined angular offsets from the thermographic camera 140 (e.g., 45 degrees and negative 45 degrees), where the angular offsets are calculated from an origin within the target examination region. Thus, when the system 100 illuminates the second subset of wall-integrated lighting elements 152 or the third subset of wall-integrated lighting elements 152 and prompts the patient to stand near to the origin of the target examination region and to face the illuminated reference location, a patient following these instructions will position themselves at an angular offset to the thermographic camera 140 equal to the angular offset of the second subset of wall-integrated lighting elements 152 and the third subset of wall-integrated lighting elements 152. In one example, the examination cell 102 includes a second subset of wall-integrated lighting elements 152 and a third subset of wall-integrated lighting elements 152 installed behind a translucent interior lining 112 that, upon illumination by the system 100, are visible to a patient within the internal examination volume.

In another implementation, the examination cell 102 includes a grid of wall-integrated lighting elements 152 arranged at various heights within the internal examination volume and at various angular offsets from the thermographic camera 140 relative to the target examination region within which the system 100 prompts the patient to locate herself. In this implementation, the system 100 can, in response to detecting noncompliance by a patient (e.g., an incorrect posture, orientation, or position), attempt to correct this noncompliance by adjusting the position of the illuminated lighting element on the wall. For example, if the system 100 detects that a patient is leaning too far forward, then the system 100 can illuminate a light higher on the interior lining 112 of the examination cell 102. The patient may then lean back in order to comply with the original instruction to face the illuminated lighting element. Thus, by illuminating lighting elements 150 arranged in a grid or in multiple locations and angular offsets, the system 100 can correct for postural, orientational, or positional biases of the patient.

More specifically, in this implementation, the examination cell includes a visual light camera arranged within the interior examination volume and defining a second field of view encompassing the target examination region within the interior examination volume. In this implementation, the computational subsystem 104 is configured to, for each target pose in the series of target poses: capture a first visual light image of the target examination region; detect a first pose of the patient in the first visual light image; in response to detecting the first pose offset from the target pose, illuminate an adjusted reference position based on an offset between the first pose and the target pose; capture a second visual light image of the target examination region; detect a second pose of the patient in the second visual light image; and, in response to detecting the second pose matching the target pose, capture the thermographic image of the patient in the target pose via the thermographic camera 140.

In yet another implementation, the examination cell 102 can include a ceiling-integrated lighting element corresponding to a target pose in the series of target poses. In this implementation, the system 100 can prompt the patient to look up toward the ceiling-integrated lighting element in order to expose her neck and thyroid gland region for imaging by the thermographic camera 140.

### 4.5.3 Projected Lighting

In one variation, as shown in FIG. 3, the examination cell 102 includes a single wide-angle digital projector 154 as the set of lighting elements 150 in order to guide the patient through the series of target poses via the illumination of various regions of the interior lining 112 of the examination cell 102. More specifically, the examination cell 102 includes a projector 154: arranged within the interior examination volume; and defining a projective region encompassing the set of reference locations. In this variation, the computational subsystem 104 is configured to, for each target pose in the series of target poses, illuminate the reference location in the set of reference locations corresponding to the target pose by projecting light toward the reference location via the projector 154. Thus, by illuminating various regions of the interior lining 112 via the wide-angle projector 154 instead of by illuminating physical lighting elements 150 installed within the internal lining, the system 100 can achieve the same functionality in guiding a patient while simplifying construction of the breast examination (e.g., by obviating the need for additional wiring and/or mounting components proximal to the internal lining).

In one implementation, the wide-angle projector 154 can be configured to project light onto the floor of the examination cell 102 in order to guide the patient in the examination cell 102 toward the target examination region within the examination cell 102 (e.g., by projecting a pair of footprints in a desired position for the patient).

In another implementation, the wide-angle projector 154 can be configured to project a structured-light pattern onto the patient for the purpose of three-dimensional reconstruction of the patient's features in order to improve a breast cancer risk assessment of the patient.

### 4.5.4 Floor-Integrated Features

In one implementation, the examination cell 102 can include a set of floor-integrated features, which may function as points of reference for a patient within the examination cell 102 such that the patient may better position themselves relative to the thermographic camera 140. More specifically, the set of floor-integrated features can include: raised bumps, indentations, edges, distinctly colored regions, or floor-integrated lighting elements that represent target poses or the target examination region within the internal examination volume. Thus, the system 100 can better prompt the patient to assume various target poses within the internal examination volume by referencing specific floor-integrated features as well as the reference positions located along the walls of the examination cell 102.

The examination cell 102 can include a floor-integrated feature indicating the position of an origin within the target examination region on which the patient is intended to stand in order to present her torso, breasts, and axillary regions within the field of view of the thermographic camera 140 of the examination cell 102. Additionally or alternatively, the examination cell 102 can include floor integrated features indicating orientations about this origin location corresponding to the series of target poses of the patient during the examination process.

In another implementation, the examination cell 102 includes a floor-integrated digital scale in order to measure and/or verify a weight of the patient. In this implementation the floor-integrated digital scale can be positioned under the origin location such that the patient is weighed without having to move to a different location in the interior examination volume. The floor-integrated digital scale can communicate with the computational subsystem 104 in order to provide any weight data of the patient recorded by the floor-integrated digital scale to the examination cell 102.

In yet another implementation, the examination cell 102 includes a floor-integrated rotating platform. In this implementation, the computational subsystem 104 is configured to, for each target pose in the series of target poses, rotate the floor-integrated rotating platform toward the reference position corresponding to the target pose. Thus, the system 100 can prompt the patient to maintain a specific pose while standing on the floor-integrated rotating platform and, instead of prompting the patient to change positions, actuate the floor-integrated rotating platform to move the patient into the target pose.

### 4.5.5 Voice Communication Subsystem

In one implementation, the examination cell 102 includes a voice communication subsystem which enables the system 100 to project pre-recorded or synthesized human speech to the patient in order to provide voice prompts (or instructions) to the patient while the patient cannot view the UI panel 130 (due to assuming target poses within the interior examination volume) and to receive voice input from the patient via speech recognition. More specifically, the voice communication subsystem can include a set of microphones and a set of speakers in communication with the computational subsystem 104; arranged in various locations within the examination cell 102; and configured to project sound into the interior examination volume and to record speech from a patient within the interior examination volume. Thus, the system 100, in response to feedback (i.e. images) recorded by the imaging subsystem, can interact with the patient to provide real-time feedback regarding the position, orientation, and/or posture of the patient within the interior examination volume. Additionally, the system 100 can issue standard instructions and prompts via the voice communication subsystem.

### 4.6 Imaging Subsystem

Generally, the examination cell 102 includes an imaging subsystem in order to record thermographic images of the patient for subsequent diagnostic analysis. More specifically, the imaging subsystem can include one or more thermographic cameras 140 and/or one or more visible-light cameras 142 in communication with the computational subsystem 104. Thus, the system 100 can: via the set of thermographic cameras 140, record thermographic images of the patient (e.g., in a series of target poses) in order to analyze these images and calculate a diagnostic assessment (e.g., a breast cancer risk assessment) for the patient and/or detect temperature anomalies within the interior examination volume; and, via the set of visible-light cameras 142, detect the position of the patient within the internal examination volume in order to determine compliance of the patient with the instructions given by the system 100 and/or to cross-reference with the thermographic images in order to generate a three-dimensional model of the patient's torso.

The imaging subsystem is connected to the computational subsystem 104 in order to coordinate recordation of thermographic and visible-light images with instructions and/or other guidance (e.g., illuminated lighting via the set of lighting elements 150, audio instructions from the voice communication subsystem, written instructions displayed on the UI panel 130) and to process these images according to Blocks of the method S100.

Generally, the imaging subsystem can include a thermographic camera 140 arranged within the interior examination volume; and defining a field of view encompassing a target examination region within the interior examination volume. More specifically, the imaging subsystem can include a set of thermographic cameras 140 suitable for clinical breast thermography such as defining an absolute resolution of at least 640 by 480 pixels, a spatial resolution of less than 0.5 milliradians, and a temperature resolution of less than 0.15 degrees Celsius. However, in variations of the imaging subsystem including multiple thermographic cameras 140, the examination cell 102 can include thermographic cameras 140 with absolute resolutions, spatial resolutions, and/or temperature resolutions outside of the aforementioned bounds. In variations include visible-light cameras 142, the examination cell 102 can include visible-light cameras 142 with wide ranges of resolutions depending on the intended use case of each of the visible-light cameras 142. Generally, for three-dimensional modelling of the patient's torso for use in diagnostics, the examination cell 102 can include a high-resolution visible-light camera 142. Alternatively, for pose and/or position detection of the patient, the examination cell 102 can include a low-resolution visible-light camera 142.

In another alternative implementation, the system 100 can include a thermographic camera or set of thermographic cameras 140 of sufficient resolution to generate a three-dimensional model of the patient's torso based on a set of thermographic images or detect a pose of the patient based on the set thermographic images. More specifically, the system 100 can, for each target pose in the series of target poses: capture a first thermographic image of the target examination region; detect a first pose of the patient in the first thermographic image; and, in response to detecting the first pose matching the target pose, store the first thermographic image of the patient for further analysis. Additionally or alternatively, the system can generate a three-dimensional model of the patient's torso based on high-resolution thermographic images (i.e., without an auxiliary visible-light camera). More specifically, in this implementation, the system can: capture a set of thermographic images of the patient in the series of target poses; and combine these images into a three-dimensional thermographic surface representing the three-dimensional distribution of the temperature of the patient's torso. Thus, the system 100 can include sufficiently high-resolution thermographic cameras to perform two- and/or three-dimensional analysis of a patient's pose and three-dimensional thermodynamic modelling of the patient's torso.

Various implementations of the examination cell 102 can include different arrangements of thermographic cameras 140 and visible-light cameras 142 such as in the stationary thermographic camera variation, the visible-light camera variation, the mobile thermographic camera variation, and the multiple thermographic camera variation, further described below.

### 4.6.1 Stationary Thermographic Camera Variation

In the stationary thermographic camera variation, the examination cell 102 includes a single stationary thermographic camera 140 positioned on the perimeter of the interior examination volume of the examination cell 102. More specifically, the examination cell 102 can include a thermographic camera 140 arranged at torso level at the perimeter of the interior examination volume and oriented parallel to the floor. Thus, by including a single stationary thermographic camera 140, the cost of the examination cell 102 can be kept comparably low (e.g., to other imaging subsystem variations), while enabling high-accuracy thermographic analysis by the system 100.

In implementations of the examination cell 102 defining a square footprint of the interior examination volume, the thermographic camera 140 can be arranged at one corner of the square footprint at torso height in the internal examination volume facing toward the center of the interior examination volume. Alternatively, in implementations of the examination cell 102 defining a circular footprint of the interior examination volume, the thermographic camera 140 can be arranged anywhere on the perimeter of the circle facing the center of the circle at torso height for an average patient. In implementations of the examination cell 102 defining a teardrop footprint, the thermographic camera 140 can be arranged at the tip of the teardrop facing the center of the teardrop.

In one implementation, the examination cell 102 can include an adjustable stand or mount for the thermographic camera 140 in order to enable a patient (or an operator) to adjust the height of the thermographic camera 140 to coincide with the patient's torso height, thereby improving thermographic image quality for the patient (e.g., reducing the incident angle between the faces of the patient's torso and the thermographic camera 140).

### 4.6.2 Visible-Light Camera Variation

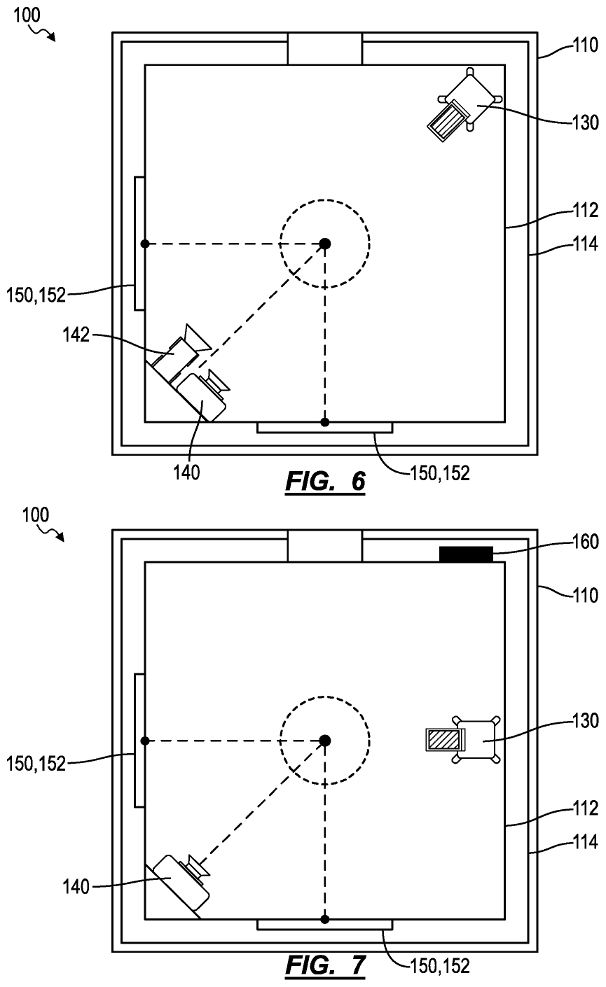

In the visible-light camera variation, shown in FIG. 6, the examination cell 102 can include a visible-light camera 142 proximal to the thermographic camera 140 in order to capture visible-light images of the interior examination volume defining a field of view aligned with the field of view of the thermographic camera 140. More specifically, the examination cell 102 can include a visual light camera: arranged within the interior examination volume; and defining a second field of view encompassing the target examination region within the interior examination volume. In this variation, the computational subsystem 104 is configured to, for each target pose in the series of target poses: capture a first visual light image of the target examination region; detect a first pose of the patient in the first visual light image; and, in response to detecting the first pose matching the target pose, capture the thermographic image of the patient in the target pose via the thermographic camera 140. Thus, in this variation, the system 100 can: record visible-light images to detect the position of the patient in the interior examination volume; capture thermographic images in response to detecting that the patient is in the correct target pose; detect an offset between the current pose of the patient and the target pose; and to cross-reference visible features of the patient with thermal features present in concurrently captured thermographic images of the patient, thereby improving the accuracy of the diagnostic assessment.

In one implementation of the visible-light camera variations, the examination cell 102 can include multiple visible-light cameras 142 in order to better capture the position of the patient within the interior examination volume and, therefore, improve feedback provided by the system 100 to the patient during the examination process.

In another implementation of the visible-light camera variation, the examination cell 102 can include a LIDAR scanner or structured light scanner in order to more accurately capture three-dimensional features of the patient. In this implementation, the computational subsystem 104 is configured to activate the LIDAR scanner and/or the structured light scanner concurrently while capturing a thermographic image via the thermographic camera. Thus, the system 100 can record a three-dimensional representation of the patient concurrent with the thermographic image of the patient in order to generate a three-dimensional thermodynamic model of the patient.

### 4.6.3 Mobile Thermographic Camera Variation

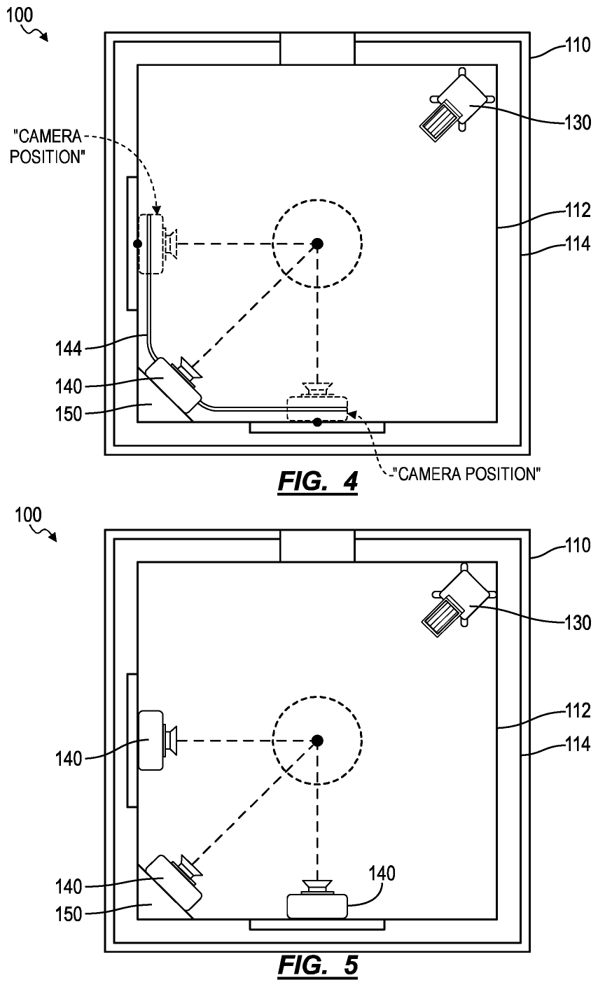

In the mobile thermographic camera variation, shown in FIG. 4, the examination cell 102 includes a thermographic camera 140 coupled to a gantry system configured to horizontally translate the thermographic camera 140, vertically translate thermographic the thermographic camera, rotate the thermographic camera 140 in pitch, and/or rotate the thermographic camera 140 in yaw in order to further minimize the incident angle defined by various surfaces of the patient's torso relative to the thermographic camera 140 and/or in order to record multiple thermographic images in quick succession in order to reconstruct a three-dimensional thermographic model of the patient, thereby improving the breast cancer risk assessment of the system 100.

In one implementation of the mobile thermographic camera variation, in response to detecting that a patient is orientated at 40 degrees relative to the thermographic camera 140 and is therefore rotationally offset from the intended orientation by five degrees, the system 100 can rotate and/or translate the thermographic camera 140 such that the patient is in the intended orientation relative to the thermographic camera 140.

In another implementation of the mobile thermographic camera variation, the system 100 can instruct the patient to stand in a single target pose and record a thermographic image of the patient and translate the thermographic camera 140 to a successive position relative to the stationary patient before recording a successive thermographic image of the patient. Thus, the system 100, by including a mobile gantry for the thermographic camera 140, can make adjustments to the field of view and/or orientation of the thermographic camera 140 to correct for incorrect positioning of the patient relative to the thermographic camera 140 and, thereby, improve the overall efficacy of the thermographic images recorded by the thermographic camera 140. Additionally, the system 100 can reduce the number of target poses that the patient assumes during the examination process by instead moving the thermographic camera 140 to obtain multiple views of the patient.

More specifically, the examination cell 102 can include a set of reference locations including a single reference location and a gantry subsystem 144 coupled to the thermographic camera 140; and configured to rotate the thermographic camera 140 about the target examination region. In this implementation, the computational subsystem 104 is configured to, for each target pose in the series of target poses: illuminate the single reference location; prompt the patient to locate within the target examination region and to orient relative to the single reference location illuminated via the set of lighting elements 150; rotate the thermographic camera 140 about the target examination region to an angular offset from the single reference location corresponding to the target pose via the gantry subsystem 144; and capture the thermographic image of the patient in the target pose via the thermographic camera 140.

In yet another implementation of the mobile thermographic camera variation, the examination cell can include a vertically mobile thermographic camera 140 such that the system 100 can detect a height of the patient (or a breast height of a patient for a thermographic breast examination) and adjust the height of the thermographic camera 140 via the gantry subsystem 144 based on the height of the patient (e.g., level with a target anatomical feature of the patient. Alternatively, the system 100 can receive an input from the patient (e.g., via the UI panel 130) and vertically adjust the height of the thermographic camera 140 via the gantry subsystem 144 based on the input.

More specifically, the examination cell 102 can include a gantry subsystem 144 coupled to the thermographic camera 140 configured to vertically translate the thermographic camera 140. In this implementation, the computational subsystem 104 is configured to: capture a first visual light image of the target examination region; detect a height of the patient in the first visual light image; access a target height of the thermographic camera 140 based on the height of the patient; and vertically translate the thermographic camera 140 to the target height.

### 4.6.4 Multiple Thermographic Camera Variation

In the multiple thermographic camera variation, shown in FIG. 5, the examination cell 102 can include a set of thermographic cameras 140 arranged about the interior examination volume of the examination cell 102, each thermographic camera 140 in the set of thermographic cameras 140 corresponding to an target pose of the patient during the examination process in order to reduce the movement required of the patient during the examination process and to reduce the elapsed time between captured thermographic images. For example, the system 100 can instruct the patient to stand in a single target pose and take multiple concurrent thermographic images of the patient via each of the set of thermographic cameras 140, thereby obtaining multiple views of the patient at multiple different orientations relative to each of the thermographic cameras 140. Thus, by including multiple thermographic cameras 140, the examination cell 102 can reduce the number of target poses that the patient assumes during the examination process by taking multiple thermographic images from different locations and orientations simultaneously.

### 4.7 Portable Variation

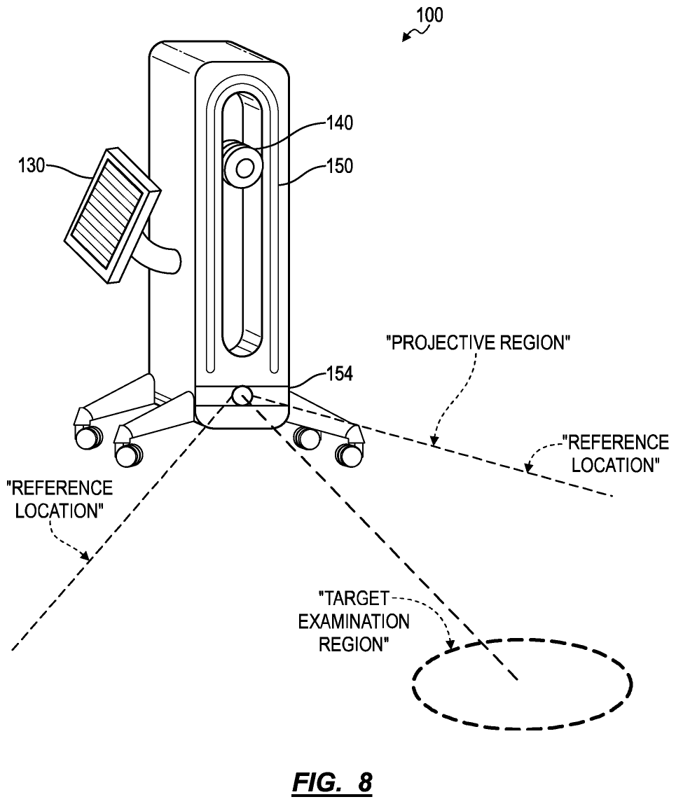

In one variation, shown in FIG. 8, the system 100 defines a portable chassis 106 including the imaging subsystem, the set of user experience elements, and components of the computational subsystem 104 further described below. In this variation, the system 100 can be positioned within any room or enclosed environment characterized by a controlled temperature with a suitable examination temperature range (e.g., between 20 and 25 degrees Celsius). Thus, the chassis 106 cooperates with the room to act as the examination cell 102.

In this variation, the chassis 106 can include a set of user experience elements including a projector 154 configured to project light onto successive reference locations (e.g., relative to the location of the chassis 106) on the floor of the room according to Blocks of the method S100, further described below. Thus, an administrator of the system 100 can move the mobile chassis to any room for execution of the method Sim, without also providing the examination cell 102.

In this variation, the chassis 106 can also include a UI panel 130 and/or speaker system in order to interface with the user, to display prompts to the user, and/or to audibly prompt the user to maintain the series of target poses. Thus, the system 100 can effectively prompt the user according to the method S100, and transiently convert a room into an examination cell 102 capable of executing an automated thermographic examination procedure.

### 5. Computational Subsystem

Generally, the system 100 can include a computational subsystem 104 configured to execute the method S100 via each of the components of the examination cell 102. More specifically, the computational subsystem 104 can include a set of local computational devices communicating directly and/or a set of remote computational devices communicating of over a network (e.g., a local or wide area network). Thus, the system 100 can execute various Blocks of the method S100 at a computational resource designed for such execution. For example, the computational subsystem 104 can include: a server configured to calculate a diagnostic assessment (e.g., a breast cancer risk assessment) by analyzing a set of thermographic images of the patient; a set of microprocessors controlling the UX elements of the examination cell 102 and/or the HVAC subsystem 120; and a tablet computer as the UI panel 130.

In one implementation, the computational subsystem 104 is configured to, following entry of a patient into the interior examination volume, for each target pose in a series of target poses: illuminate a reference location in the set of reference locations via the set of lighting elements 150, the reference location corresponding to the target pose; prompt the patient to locate within the target examination region and to orient relative to the reference location illuminated via the set of lighting elements 150; and capture a thermographic image of the patient in the target pose via the thermographic camera 140. The computational subsystem 104 is further configured to generate a diagnostic assessment for the patient based on a set of thermographic images comprising the thermographic image of the patient in the target pose for each target pose in the series of target poses.

### 6. Onboarding

Generally, the system 100 can, upon entry of a patient into the interior examination volume, receive personal details and/or medical and clinical history information from patient via the UI panel 130. More specifically, the system 100 can: receive a series of inputs from a patient indicating personal details of the patient and a medical and clinical history information of the patient; and generate a patient profile corresponding to the patient and representing the personal details of the patient and the medical history information of the patient. Thus, the system 100 can receive data from the patient in order to inform execution of Blocks of the method S100 and to improve the accuracy of the breast cancer risk assessment calculated by the system 100 based on the medical history information of the patient.

In one implementation, instead of utilizing the UI panel 130, the system 100 can ask a series of scripted questions and record vocal responses of the patient via the voice communication subsystem. Thus, the system 100 can create a more natural experience for the patient.

The system 100 can prompt the patient to input personal details such as a height, a weight, and a breast size of the patient in order to better adjust the location of the thermographic camera 140 (e.g., via a gantry system or via manually adjustment by an operator of the system 100 or by the patient herself) prior to initiating the examination process. Additionally, the system 100 can prompt the patient to enter relevant medical history regarding her breast health such as her family history of breast cancer, her BRCA1 and BRCA2 results, her own history of breast cancer diagnoses, her history of mammectomy, etc. in order to provide additional data with which the system 100 can calculate a breast cancer risk assessment for the patient.

### 7. Acclimatization

Generally, upon entry of the patient into the interior examination volume of the examination cell 102, the system 100 can instruct the patient to disrobe (e.g., via the UI panel 130 or the voice communication subsystem) and concurrently lower the temperature of the interior examination volume via the HVAC subsystem 120 in order to allow the patient to acclimatize to the environment of the interior examination volume characterized by the predetermined examination temperature range. More specifically, the breast examination system 100 can: prompt the patient in the interior examination volume to disrobe in Block S110; and set the temperature of the interior examination volume to within the predetermined examination temperature range via the HVAC subsystem 120 in Block S112. Thus, the system 100 can improve the efficacy of the thermographic images in the calculation of the patient's breast cancer risk assessment by allowing a thermal equilibrium to develop between the skin of the patient's torso and the environment of the interior examination volume.

In one implementation, the system 100 can execute the abovementioned acclimatization steps, while prompting the patient to input her personal details and/or medical history as described above, thereby onboarding and acclimatizing the patient concurrently.

Upon initiating temperature control of the interior examination volume, the system 100 can initiate a timer of a predetermined acclimatization duration and initiate subsequent Blocks of the method S100 upon expiration of this timer. For example, the system 100 can allow the patient to acclimatize for ten minutes prior to proceeding with subsequent Blocks of the method S100.

### 7.1 Acclimatization Detection

In one variation of the method S100, the system 100 can detect whether the skin of the patient has reached thermal equilibrium with the environment of the internal examination volume in order to ensure that the system 100 only initiates subsequent Blocks of the method S100 after this has occurred, thereby improving the sensitivity of the breast cancer risk assessment and reducing the incidence of anomalous temperature readings in thermographic images recorded by the system 100. More specifically, the system 100 can: record successive thermographic images of the patient and measure a temperature variation of one or more regions of the patient's torso between images; and, in response to a threshold temperature variation exceeding the temperature variation between thermographic images, the system 100 can initiate subsequent Blocks of the method S100. Thus, because of variations in capillary action between patients, the system 100 can adaptively wait for complete acclimatization of each patient to the environment of the interior examination volume of the examination cell 102.

In one implementation, the system 100 can also detect temperature anomalies within the interior examination volume by: recording thermographic images of the interior examination volume; comparing temperatures of various surfaces of the interior lining 112; and detecting temperature gradients on these surfaces. In this implementation, the system 100 can initiate subsequent Blocks of the method S100, in response to detecting a maximum magnitude of temperature gradients on the interior lining 112 of the examination cell 102 less than a threshold magnitude. Additionally, in response to detecting a temperature gradient on a particular surface or between surfaces of the interior lining 112 of the examination cell 102, the system 100 can redirect airflow via the HVAC subsystem 120 to reduce these temperature gradients.

### 7.2 Dynamic Thermography

In one implementation, the system 100 can, during the acclimatization process described above: initiate a cold shock within the examination cell 102; prompt the patient to assume a target pose; and capture a series of thermographic images of the patient in the target pose. Thus, in this implementation, the system 100 can capture multiple successive thermographic images that depict the patient's vascular reaction to a cold shock. The system 100 can subsequently execute dynamic thermography techniques to analyze this series of thermographic images while generating the diagnostic assessment as is further described below.

### 8. Target Poses

Upon completion of the above-described onboarding and acclimatization Blocks, the system 100 can successively prompt the patient to assume a series of positions relative to the thermographic camera 140 in order to record multiple images of the patient's torso from various angles, thereby improving the efficacy of a breast cancer risk assessment. More specifically, the system 100 can: at a second time succeeding the first time by an acclimatization duration, prompt the patient to locate to the target examination region in Block S120; and, for each target pose in the series of target poses, illuminate a reference location in the set of reference locations via the set of lighting elements 150, the reference location corresponding to the target pose in Block S130. The system 100 can then prompt the patient to orient relative to the reference location illuminated via the set of lighting elements 150 in Block S140. In particular, the system 100 can: prompt the patient to stand straight with her hands placed on the back of her head and face toward an illuminated lighting element 150 (or other indicator) on the interior lining 112 of the examination cell 102 to adopt a first target pose. The system 100 can then change the position of this lighting element 150 (or other indicator) or issue further instructions to face a different indicator or a different direction to prompt the patient into assuming a second target pose. The system 100 can continue as such until the patient has assumed the entire series of target poses and the intended set of thermographic images have been recorded. Thus, the system 100 is able to administer a multi-orientation thermographic examination (e.g., a thermographic breast examination) without the intervention of medical or other personnel.

In one implementation, the system 100 can prompt the patient to assume three positions by: prompting the patient to face an illuminated lighting element; at a first time, illuminating a first lighting element proximal to the thermographic camera 140; at a second time, deactivating the first lighting element and illuminating a second lighting element angularly offset from the thermographic camera 140 by positive 45 degrees (at the location of the patient in the interior examination volume); and, at a third time, deactivating the second lighting element and illuminating a third lighting element angularly offset from the thermographic camera 140 by negative 45 degrees. Thus, the system 100 can record three thermographic images of the patient in three orientations relative to the thermographic camera 140 by recording images of the patient in coordination with the illumination and deactivation of each lighting element. For example, the system 100 can: record a first thermographic image between the first time and the second time; a second thermographic image between the second time and the third time; and a third thermographic image after the third time.

More specifically, the examination volume can define a set of reference locations including: a first reference position aligned with the thermographic camera 140 relative to the target examination region; a second reference position angularly offset from the thermographic camera 140 by forty-five degrees in a first direction relative to the target examination region; and a third reference position angularly offset from the thermographic camera 140 by forty-five degrees in a second direction opposite the first direction relative to the target examination region. The system 100 can then successively illuminate each of these reference positions and record a corresponding thermographic image as follows.

For a first target pose in the series of target poses, the system 100 can: illuminate the first reference location in the set of reference locations via the set of lighting elements 150; prompt the patient to locate within the target examination region and to orient relative to the first reference location illuminated via the set of lighting elements 150; and capture a first thermographic image of the patient in the first target pose via the thermographic camera 140. For a second target pose in the series of target poses, the system 100 can: illuminate the second reference location in the set of reference locations via the set of lighting elements 150; prompt the patient to locate within the target examination region and to orient relative to the second reference location illuminated via the set of lighting elements 150; and capture a second thermographic image of the patient in the second target pose via the thermographic camera 140. For a third target pose in the series of target poses, the system 100 can: illuminate the third reference location in the set of reference locations via the set of lighting elements 150; prompt the patient to locate within the target examination region and to orient relative to the third reference location illuminated via the set of lighting elements 150; and capture a third thermographic image of the patient in the third target pose via the thermographic camera 140. The system can then generate the diagnostic assessment for the patient based on the set of thermographic images comprising the first thermographic image, the second thermographic image, and the third thermographic image, as is further described below.

### 8.1 Target Pose Adjustment and Verification

Generally, the system 100 can adaptively communicate with the patient in real-time in order to modify the pose of the patient to match a target pose in preparation for capture of a thermographic image, thereby improving the frequency with which the system 100 captures images of the patients in the target poses. More specifically, the system 100 can: capture an image (either thermographic or visible-light image) of a patient assuming a first target pose; detect an actual position of the patient based on the image; and, in response to detecting a deficiency between the first target pose and the actual position of the patient, prompt the patient to correct the deficiency. Thus, the system 100 can issue audio commands (via the voice communication subsystem) and visual commands (via the set of lighting elements 150 or the wide-angle projector 154) to correct the position of a patient in real-time prior to recording a thermographic image of the patient.

The system 100 can identify deficiencies, via a computer vision algorithm, such as incorrect posture (slouching or arching of the patient's back), incorrect orientation (an rotational offset between the orientation of the patient in the target pose and the orientation of the patient in the actual position), and incorrect position (a translational offset between the position of the patient in the target pose and the position of the patient in the actual position). In one example, the system 100 can simulate a current target pose of the patient for a patient of similar height and weight to the patient and compare the actual position of the patient to this simulated position.

In one implementation, upon identifying a deficiency in the position of the patient, the system 100 can: adjust the position of an illuminated lighting element or illuminated region on the interior lining 112; and continue monitoring the position (e.g., via a series of additional images) of the patient as she responds to this change in lighting until the actual position of that patient matches the target pose of the patient. For example, the system 100 can change the height of the illuminating lighting element in order to mitigate a slouching deficiency of the patient. In an alternative example, the system 100 can change the rotational offset of the illuminated lighting element in order to signal additional rotation of the patient relative to the thermographic camera 140.

In another implementation, upon identifying a deficiency in the position of the patient, the system 100 can: generate an audio message indicating the deficiency in the position of the patient to the patient and prompt the patient to adjust the deficiency. For example, the system 100 can generate an audio message such as “please stand straight and face the light” in response to detection of a slouching posture. In an alternative example, the system 100 can generate an audio message such as “please rotate toward the light” in response to detecting angular offset deficiency.

In another implementation, the system 100 can indicate compliance of the patient with the current target pose in response to detecting less than a threshold deficiency of the actual position of the patient when compared to the current target pose of the patient. For example, the system 100 can issue a confirmatory audio message such as “please remain still while the image is recorded” or can modify the color of the lighting (e.g., turn the lighting green) in order to indicate to the patient that they are in compliance with the target pose in the examination process.

### 9. Thermographic Image Capture

Generally, upon prompting the patient to assume an target pose in the series of target poses and/or detecting that the patient has assumed an target pose in the series of target poses, the system 100 can record a thermographic image of the patient. More specifically, the system 100 can capture a thermographic image of the patient via the thermographic camera 140 in Block S150. In particular, for a thermographic breast examination, the system 100 can record a thermographic image depicting the torso of the patient and corresponding to each target pose in a series of target poses. Thus, the system 100 can record a set of thermographic images for calculation of the breast cancer risk assessment of the patient. Additionally, the system 100 can record thermographic images depicting the breast, torso, and/or axillary region of the patient.

In one implementation, the system 100 can: record a first thermographic image depicting the patient facing the thermographic camera 140; record a second thermographic image depicting the patient facing positive 45 degrees relative to the thermographic camera 140; and a third thermographic image depicting the patient facing negative 45 degrees relative to the thermographic camera 140. However, the system 100 can record additional or fewer thermographic images depending on the implementation.

In another implementation, the system 100 can, prior to recording a thermographic image: record the temperature of the interior examination volume via a set of temperature sensors arranged throughout the interior examination volume and record the humidity of the interior examination volume via a humidity sensor arranged within the interior examination volume; and calibrate the thermographic camera 140. Thus, the system 100 can adjust the settings of the thermographic camera 140 based on the temperature and humidity within the interior examination volume prior to recording a thermographic image, thereby improving the temperature accuracy of each thermographic image.

### 9.1 Multiple Thermographic Image Camera Capture

In the mobile thermographic camera variation and the multiple thermographic camera variation, the system 100 can record multiple images of a patient in the same target pose and can stitch or otherwise reconstruct these images into a single (two- or three-dimensional) representation of the patient for the purpose of calculating the breast cancer risk assessment of the patient.

In the mobile thermographic camera variation, in one implementation, the system 100 can move the thermographic camera 140 in a predetermined pattern while recording a series of thermographic images in order to change the orientation of the thermographic camera 140 relative to various surfaces of the patient's torso, thereby obtaining more accurate temperature estimates of these surfaces by extracting temperature data from regions of the patient's torso that are estimated to be substantially perpendicular to the orientation of the thermographic camera 140 in each thermographic image in the series of thermographic images.

In the mobile thermographic camera variation, in another implementation, the system 100 can translate and/or rotate the thermographic camera 140 to multiple distinct locations throughout the interior examination volume in order to record a set of thermographic images corresponding to each target pose of the patient relative to the thermographic camera 140 without prompting the patient to assume each of these target poses (instead the patient assumes a single target pose relative to the thermographic camera 140 and the camera moves to a series of positions and orientations relative to the patient).

In the multiple thermographic camera variation, the system 100 can record a thermographic image from each of the set of thermographic cameras 140 concurrently, thereby recording thermographic images of the patient from multiple perspectives at the same time and without prompting the patient to assume multiple target poses.

### 9.2 Thermographic Image Verification

In one implementation, upon recording each thermographic image, the system 100 can verify whether the thermographic image does not contain anomalies; and, in response to detecting that the thermographic image does contain anomalies, rerecord the thermographic images. More specifically, the system 100 can: access a set of usability criteria; evaluate the set of thermographic images based on the set of usability criteria; and, in response to detecting that a subset of thermographic images in the set of thermographic images fail a usability criterium in the set of usability criteria, recapture the subset of thermographic images. Thus, the system 100 can reduce the frequency of recording unusable images, thereby reducing the rate at which it is necessary for the system 100 to reexamine patients.

In one implementation, the system 100 can verify thermographic images by executing a breast mask model on the thermographic images. In this implementation, the system 100 can execute a breast mask model (e.g., a convolution artificial neural network or other machine learning model) that has been trained to identify regions of a thermographic image depicting a human's breast and axillary regions for the purpose of breast cancer risk assessment. The system 100 can execute this breast mask model and, in response to the breast mask model failing to detect a contiguous region in the thermographic image corresponding to the patient's breast region and axillary region, the system 100 can rerecord the thermographic image.

In another implementation, the system 100 can detect that the patient has not fully disrobed by detecting temperatures below a predetermined threshold within a breast region of the thermographic image. In this implementation, the system 100 can prompt the patient to fully disrobe (e.g., via an audio message) and rerecord the thermographic image.

Additionally or alternatively, the system 100 can identify infrared reflection from interior surfaces of the examination cell 102, by detecting regions of elevated temperature above a predetermined threshold on the torso of the patient and/or by detecting an interior surface characterized by an elevated temperature directly in the thermographic image. In this implementation, the system 100 can configure the HVAC subsystem 120 to reduce the temperature of the detected interior surface characterized by the elevated temperature; and, in response to detecting that the temperature of the interior surface is within the predetermined threshold temperature, rerecord the thermographic image.

In yet another implementation, the system 100 can detect blur in the image caused by movement of the patient during the exposure duration of the thermographic camera 140. In this implementation, the system 100 can: prompt the patient (via an audio message) to remain still while the image is recorded; and rerecord the thermographic image.

### 10. Diagnostics

Generally, following capture of the set of thermographic images, the system 100 can generate a diagnostic assessment for the patient based on a set of thermographic images including the thermographic image of the patient in each target pose in the series of target poses in Block S160. The system 100 can generate a diagnostic assessment indicating a level of risk for an oncological condition, such as breast cancer or thyroid cancer. Additionally or alternatively, the system 100 can generate a diagnostic assessment identifying the presence (or absence) of abnormalities (e.g., abnormal masses or vascular patterns). Furthermore, the system 100 can generate a diagnostic assessment that indicates a binary result representing either a normal result or a referral to a physician for further examination. Thus, the system 100 can generate diagnostic assessments with varying levels of specificity based on the application and/or regulatory environment of the system 100 and can coordinate with physicians in order to deliver a complete diagnosis.

In one implementation, the system 100 can generate a breast cancer risk assessment based on the set of thermographic images. For example, upon capturing a set of thermographic images, the system 100 can execute a breast cancer risk assessment model (e.g., a machine learning model) on the set of thermographic images in order to calculate a breast cancer risk assessment of the patient. Alternatively, the system 100 can transmit the set of thermographic images to a remotely located physician for further evaluation of the set of thermographic images. Thus, the system 100 can provide a near-real-time breast cancer risk assessment by processing the set of thermographic images recorded during the examination process as the patient dresses and exits the examination cell 102. The system 100 can transmit the calculate breast cancer risk assessment directly to the patient via a provided point of contact (e.g., phone number, email address). Alternatively, the system 100 can transmit the breast cancer risk assessment to an operator of the system 100 seated outside of the examination cell 102, who may then communicate this breast cancer risk assessment to the patient as she leaves the examination cell 102.

In one variation, the system 100 can calculate a breast cancer risk assessment for the patient based on the set of thermographic images by executing a machine learning model. The machine learning model can include a neural network (e.g., a convolutional neural network, a recurrent neural network, a long short-term memory neural network) or any other statistical method for classification of the set of thermographic images of the patient. More specifically, the system 100 can define an input tensor representative of the set of thermographic images. In one implementation, the system 100 can generate a three-dimensional representation of the torso of the patient based on the set of thermographic images and convert this three-dimensional representation into an input tensor for input to the machine learning model.

In one implementation, the machine learning model outputs a binary score indicating further tests are necessary or that the patient has a low risk of breast cancer. Alternatively, the machine learning model outputs a risk factor or risk categorization based on the input temperature data (e.g., such as a BI-RADS score).

Additionally or alternatively, the machine learning model can display the set of thermographic images (or the 3D model) of the patient's torso and highlight the location of potentially cancerous tissue. Furthermore, the system 100 can: execute a 3D thermal simulation of the internal volume of the patient's torso (e.g., via finite element analysis); render this 3D thermal simulation; and input this 3D thermal simulation into a machine learning model to estimate the size, location, and metabolic activity of a lesion or tumor inside the breast. More specifically, the system 100 can: for each target pose in the set of target poses, capture a visual light image of the patient via a visual light camera arranged in the examination cell 102 concurrent with the thermographic image of the patient; generate a three-dimensional model of a torso of the patient based on a set of visual light images comprising the visual light image for each target pose in the set of target poses; and simulate a three-dimensional thermodynamic model based on the three-dimensional model of the torso of the patient and the set of thermographic images; and generate the diagnostic assessment for the patient based on the three-dimensional thermodynamic model.

### 10.1 Physician Portal

In one implementation, the system 100 can generate a diagnostic assessment by: transmitting the set of thermographic images to a physician portal for evaluation by a physician; and receive input at the physician portal indicating the diagnostic assessment. In this implementation, the system 100 cooperates with an application executed on a physician's electronic device in order to present to a physician: the set of thermographic images of a patient captured by the system 100 during the thermographic examination process, a patient's health data input to the system 100 during the thermographic examination process (e.g., via the UI panel 130), and/or the patient's relevant electronic medical records (e.g., from an online database). The physician portal can then receive an input from the physician indicating a diagnostic assessment based on the presented images and information. Thus, the system 100, via the physician portal, can transmit thermographic images and related health data to a remote physician and receive a diagnostic assessment for the patient in near-real time.

In one example, the system can: transmit the set of thermographic images to a physician portal for evaluation by a physician; and receive input at the physician portal indicating the breast cancer risk assessment. Additionally, in this example, the system 100 can receive input from the physician indicating a location and description of any abnormalities present in the set of thermographic images of the patient.

### 11. Reporting

Generally, upon generating a diagnostic assessment for a patient based on the set of thermographic images, the system 100 can report the diagnostic assessment to the patient directly in real-time (e.g., via the UI panel 130 within the examination volume) or post-examination via cooperation with a patient portal (e.g., an application) executing on a user device of a patient. More specifically, the system 100 can: receive electronic contact information for the patient; and transmit the diagnostic assessment to an electronic device of the patient based on the electronic contact information. Thus, the system 100 can communicate the results of the autonomously administered thermographic examination in real or near-real time upon completion of the thermographic examination process.

In one implementation, the system 100 cooperates with the patient portal to present a summary of the diagnostic assessment to the patient. In this implementation, the summary of the diagnostic assessment can include a high-level overview of the results indicating a normal or abnormal diagnosis, a risk category (e.g., a BI-RADS score), or an indication of whether a follow up with a physician is recommended. In applications of the system 100 in which the system 100 executes a thermographic breast examination, the system 100 can further present, via the patient portal, a breast-specific analysis of each breast of the patient. The breast-specific analysis can include thermographic images of each breast of the patient, graphical indications of identified abnormal masses within each breast, and/or written explanations for the diagnostic assessment communicated via the diagnostic report.

In another implementation, the system 100 can, in response to generating a diagnostic assessment recommending a follow up visit to physician, transmit a report including the diagnostic assessment and the set of thermographic images to a physician portal of the patient's physician in anticipation of a visit to the physician by the patient.

The systems and methods described herein can be embodied and/or implemented at least in part as a machine configured to receive a computer-readable medium storing computer-readable instructions. The instructions can be executed by computer-executable components integrated with the application, applet, host, server, network, website, communication service, communication interface, hardware/firmware/software elements of a user computer or mobile device, wristband, smartphone, or any suitable combination thereof. Other system 100s and methods of the embodiment can be embodied and/or implemented at least in part as a machine configured to receive a computer-readable medium storing computer-readable instructions. The instructions can be executed by computer-executable components integrated by computer-executable components integrated with apparatuses and networks of the type described above. The computer-readable medium can be stored on any suitable computer readable media such as RAMs, ROMs, flash memory, EEPROMs, optical devices (CD or DVD), hard drives, floppy drives, or any suitable device. The computer-executable component can be a processor but any suitable dedicated hardware device can (alternatively or additionally) execute the instructions.

As a person skilled in the art will recognize from the previous detailed description and from the figures and claims, modifications and changes can be made to the embodiments of the invention without departing from the scope of this invention as defined in the following claims.

I claim:

1. A system for thermographic examination comprising:
an examination cell comprising:
an exterior enclosure defining an interior examination volume;
a thermographic camera:
arranged within the interior examination volume; and
defining a field of view encompassing a target examination region within the interior examination volume; and
a set of lighting elements configured to illuminate a set of reference locations within the interior examination volume; and
a computational subsystem configured to, following entry of a patient into the interior examination volume:
for each target pose in a series of target poses:
illuminate a reference location in the set of reference locations via the set of lighting elements, the reference location corresponding to the target pose;
prompt the patient to locate within the target examination region and to orient relative to the reference location illuminated via the set of lighting elements; and
capture a thermographic image of the patient in the target pose via the thermographic camera; and
generate a diagnostic assessment for the patient based on a set of thermographic images comprising the thermographic image of the patient in the target pose for each target pose in the series of target poses.

2. The system of claim 1, wherein the examination cell comprises an interior lining:
arranged within the interior examination volume;
defining a plenum space between the interior lining and the exterior enclosure; and
characterized by low infrared reflectivity.

3. The system of claim 2:
wherein the examination cell comprises a heating, ventilation, and air conditioning subsystem configured to force temperature-controlled air into the plenum space; and
wherein the computational subsystem is further configured to control a temperature of the interior examination volume within an examination range of temperatures via the heating ventilation and air conditioning subsystem.

4. The system of claim 2, wherein the examination cell comprises an intermediate enclosure defining an insulating layer between the interior lining and the exterior enclosure.

5. The system of claim 2:
wherein the set of lighting elements comprises a set of wall-integrated lighting elements:
arranged at the set of reference locations; and
integrated with the interior lining beneath an interior surface of the interior lining; and
wherein the computational subsystem is further configured to, for each target pose in the series of target poses, illuminate the reference location in the set of reference locations by activating a subset of wall-integrated lighting elements in the set of wall-integrated lighting elements, the subset of wall-integrated lighting elements arranged at the reference location.

6. The system of claim 1:
wherein the set of lighting elements comprises a projector:
arranged within the interior examination volume; and
defining a projective region encompassing the set of reference locations; and
wherein the computational subsystem is further configured to, illuminate the reference location in the set of reference locations by projecting light toward the reference location via the projector.

7. The system of claim 1:
wherein the examination cell comprises a user-interface panel:
arranged within the interior examination volume; and
comprising a display;
wherein the computational subsystem is further configured to, for each target pose in the series of target poses, locate within the target examination region and to orient relative to the reference location, illuminated via the set of lighting elements, via the display of the user-interface panel.

8. The system of claim 1:
wherein the examination cell comprises a visual light camera:
arranged within the interior examination volume; and
defining a second field of view encompassing the target examination region within the interior examination volume; and
wherein the computational subsystem is further configured to, for each target pose in the series of target poses:
capture a first visual light image of the target examination region;
detect a first pose of the patient in the first visual light image; and
in response to detecting the first pose matching the target pose, capture the thermographic image of the patient in the target pose via the thermographic camera.

9. The system of claim 1:
wherein the examination cell comprises a visual light camera:
arranged within the interior examination volume; and
defining a second field of view encompassing the target examination region within the interior examination volume; and
wherein the computational subsystem is further configured to, for each target pose in the series of target poses:
capture a first visual light image of the target examination region;
detect a first pose of the patient in the first visual light image;
in response to detecting the first pose offset from the target pose, illuminate an adjusted reference position based on an offset between the first pose and the target pose;
capture a second visual light image of the target examination region;
detect a second pose of the patient in the second visual light image; and
in response to detecting the second pose matching the target pose, capture the thermographic image of the patient in the target pose via the thermographic camera.

10. The system of claim 1:
wherein the examination cell comprises a gantry subsystem coupled to the thermographic camera configured to vertically translate the thermographic camera; and
wherein the computational subsystem is further configured to:
capture a first visual light image of the target examination region;
detect a height of the patient in the first visual light image;
access a target height of the thermographic camera based on the height of the patient; and
vertically translate the thermographic camera to the target height.

11. The system of claim 1:
wherein the set of reference locations comprises:
a first reference position aligned with the thermographic camera relative to the target examination region;
a second reference position angularly offset from the thermographic camera by forty-five degrees in a first direction relative to the target examination region; and
a third reference position angularly offset from the thermographic camera by forty-five degrees in a second direction opposite the first direction relative to the target examination region; and
wherein the computational subsystem is further configured to:
for a first target pose in the series of target poses:
illuminate the first reference location in the set of reference locations via the set of lighting elements;
prompt the patient to locate within the target examination region and to orient relative to the first reference location illuminated via the set of lighting elements; and
capture a first thermographic image of the patient in the first target pose via the thermographic camera;
for a second target pose in the series of target poses:
illuminate the second reference location in the set of reference locations via the set of lighting elements;
prompt the patient to locate within the target examination region and to orient relative to the second reference location illuminated via the set of lighting elements; and
capture a second thermographic image of the patient in the second target pose via the thermographic camera;
for a third target pose in the series of target poses:
illuminate the third reference location in the set of reference locations via the set of lighting elements;
prompt the patient to locate within the target examination region and to orient relative to the third reference location illuminated via the set of lighting elements; and
capture a third thermographic image of the patient in the third target pose via the thermographic camera; and
generate the diagnostic assessment for the patient based on the set of thermographic images comprising the first thermographic image, the second thermographic image, and the third thermographic image.

12. The system of claim 1:
wherein the set of reference locations comprises a single reference location;
wherein the examination cell comprises a gantry subsystem:
coupled to the thermographic camera; and
configured to rotate the thermographic camera about the target examination region; and
wherein the computational subsystem is further configured to, for each target pose in the series of target poses:
illuminate the single reference location;
prompt the patient to locate within the target examination region and to orient relative to the single reference location illuminated via the set of lighting elements;
rotate the thermographic camera about the target examination region to an angular offset from the single reference location corresponding to the target pose via the gantry subsystem; and
capture the thermographic image of the patient in the target pose via the thermographic camera.

13. The system of claim 1:
wherein the examination cell comprises a wall-integrated black body radiator arranged opposite the thermographic camera; and
wherein the computational subsystem is further configured to:
capture a calibration image via the thermographic camera depicting the wall-integrated black body radiator; and
calibrate the thermographic camera based on the calibration image.

14. A method for administering a thermographic examination executed at an examination cell comprising an exterior enclosure defining an interior examination volume, a thermographic camera arranged within the interior examination volume and defining a field of view encompassing a target examination region within the interior examination volume, and a set of lighting elements configured to illuminate a set of reference locations within the interior examination volume, the method comprising:
at first time, prompting a patient in the interior examination volume to disrobe;
at a second time succeeding the first time by an acclimatization duration, prompting the patient to locate to the target examination region;
for each target pose in a series of target poses:
illuminating a reference location in the set of reference locations via the set of lighting elements, the reference location corresponding to the target pose;
prompting the patient to orient relative to the reference location illuminated via the set of lighting elements; and
capturing a thermographic image of the patient via the thermographic camera; and
generating a diagnostic assessment for the patient based on a set of thermographic images comprising the thermographic image of the patient for each target pose in the series of target poses.

15. The method of claim 14, wherein generating the diagnostic assessment for the patient based on the set of thermographic images comprises generating a breast cancer risk assessment based on the set of thermographic images.

16. The method of claim 15, wherein generating a breast cancer risk assessment based on the set of thermographic images comprises:
executing a breast cancer risk assessment model based on the set of thermographic images; and
calculating the breast cancer risk assessment based on an output of the breast cancer risk assessment model.

17. The method of claim 15, wherein generating a breast cancer risk assessment based on the set of thermographic images comprises:
transmitting the set of thermographic images to a physician portal for evaluation by a physician; and
receiving input at the physician portal indicating the breast cancer risk assessment.

18. The method of claim 14, further comprising:
receiving electronic contact information for the patient; and
transmitting the diagnostic assessment to an electronic device of the patient based on the electronic contact information.

19. The method of claim 14, further comprising:
accessing a set of usability criteria;
evaluating the set of thermographic images based on the set of usability criteria; and
in response to detecting that a subset of thermographic images in the set of thermographic images fail a usability criterium in the set of usability criteria, recapturing the subset of thermographic images.

20. The method of claim 14:
further comprising:
for each target pose in the set of target poses, capturing a visual light image of the patient via a visual light camera arranged in the examination cell concurrent with the thermographic image of the patient;
generating a three-dimensional model of a torso of the patient based on a set of visual light images comprising the visual light image for each target pose in the set of target poses; and
simulating a three-dimensional thermodynamic model based on the three-dimensional model of the torso of the patient and the set of thermographic images; and
wherein generating the diagnostic assessment for the patient based on the set of thermographic images comprises generating the diagnostic assessment for the patient based on the three-dimensional thermodynamic model.
